(19) United States
(12) Patent Application Publication
(10) Pub. No.: US 2025/0255505 A1
(43) Pub. Date: Aug. 14, 2025

(54) SYSTEM AND METHOD FOR USING BLOOD FLOW MEASUREMENTS TO MONITOR HEALTH FUNCTIONS

(71) Applicant: Guy P. Curtis, San Diego, CA (US)

(72) Inventor: Guy P. Curtis, San Diego, CA (US)

(21) Appl. No.: 18/438,440

(22) Filed: Feb. 10, 2024

(51) Int. Cl. A61B 5/0295; A61B 5/00; A61B 5/0205; A61B 5/021; A61B 5/024; A61B 5/1455; A61B 7/04

(52) U.S. Cl. CPC A61B 5/0295; A61B 5/0205; A61B 5/7246; A61B 5/7278; A61B 5/021; A61B 5/024; A61B 5/14551; A61B 7/04

(57) ABSTRACT

A system and method for monitoring a patient's blood flow waveform require a pulse oximeter to non-invasively measure changes in parametric and blood flow measurements of the waveform. These parametric measurements, namely changes in diastolic pressure, ±Δp_{d}, changes in systolic pressure, ±Δpₛ, and changes in pulse time rate, ±Δtᵣ, collectively affect the patient's blood flow volume. Thus, they can also be used, collectively, to assess the patient's heart muscle function as an indicator of his/her health condition.

### FIELD OF THE INVENTION

The present invention pertains generally to systems and methods for analyzing a patient's blood flow waveform to measure aspects of his/her functioning circulatory system. More specifically, the present invention pertains to a system and method for continuously evaluating a plurality of parametric measurements of a patient's blood flow waveform to evaluate the patient's blood pressure vascular resistance to flow and heart function. The present invention is particularly, but not exclusively, useful for evaluating changes in the parametric measurements of consecutive waveforms, based on blood flow volume, to assess the condition of a patient's cardio vasculature system.

### BACKGROUND OF THE INVENTION

It is routine in clinical practice to measure a patient's peak systolic pressure together with his/her comparable diastolic pressure during a heartbeat. A comparison of these two measurements is thereafter typically referred to as the patient's “blood pressure”. Although this “blood pressure” has been considered sufficient for diagnostic purposes, these two pressure measurements describe only part of a patient's blood flow waveform. In fact, the patient's blood flow waveform from which “blood pressure” is measured provides much more additional information of clinical value than has thus far been exploited.

From a technical perspective, the blood flow waveform is a trace of the continuously changing amplitude and location of blood pressure measurements over a measurable time. Moreover, the “blood pressure” identified above is a mere snapshot of this trace. Specifically, in the snapshot there is no consideration of time. Thus, other valuable information concerning a blood pressure waveform of a patient's heart muscle function has been ignored from a dynamic perspective.

In an evaluation of a patient's heart muscle function, the concept of time requires a dynamic perspective of the blood pressure waveform in a pulse-to-pulse comparison. For instance, time considerations set a patient's pulse rate. Further, a time sequence of pulsatile blood flow waveforms invites dynamic considerations of changes in the extremes of diastolic pressure and systolic pressure. Moreover, the time rate of pressure changes between these pressure extremes during each pulse duration is of diagnostic value. For purposes of this disclosure, all of the variables that are involved in defining a blood pressure waveform are hereinafter collectively referred to as “parametric measurements”.

As recognized for the present invention, an appreciation of the parametric measurements that define a patient's blood flow waveform, and how these parametric measurements change with time in consecutive waveforms of blood flow, can be analyzed in terms of changes in the volume of blood flow as evidenced by the blood flow waveform. This is so because it is the parametric measurements that effectively determine a blood flow waveform. Furthermore, parametric measurements also provide valuable insight into the patient's cardiac performance and peripheral resistance to blood flow in the arteries of the patient.

For the reasons set forth above, it is an object of the present invention to underscore the importance of evaluating various additional factors that will result from an analysis of a patient's blood flow waveform. Another object of the present invention is to evaluate these additional factors for further consideration and use in a comprehensive clinical diagnosis. Yet another object of the present invention is to emphasize the clinical benefits which result from a dynamic evaluation of consecutive blood flow waveforms that are based on parametric measurements of a blood flow waveform. Still another object of the present invention is to provide a blood flow monitor capable of performing the above-cited objects which is easy to use, is simple to manufacture, and is comparatively cost effective.

### SUMMARY OF THE INVENTION

A blood pressure monitor in accordance with the present invention collects parametric measurements form a patient's blood pressure waveform that can be used to assess and evaluate a patient's health condition. Importantly, these parametric measurements are taken from blood pressure values that essentially define a blood flow waveform from a patient. These measurements are then compared with those of both prior and subsequently measured waveforms. This comparison thus provides a basis for a more comprehensive diagnosis of a patient's health condition based on consecutively obtained blood flow pressure measurements.

The present invention also recognizes that a pulse oximeter will provide information regarding the patient's arterial oxygen saturation, and additional information comparable to a patient's blood pressure waveform. Specifically, the present invention recognizes that raw data from the red and infrared diodes of a pulse oximeter can be displayed as a waveform which is comparable to the intra-arterial blood pressure obtained from a large artery using a sphygmomanometer. The importance of this recognition is that a pulse oximeter effectively imitates a patient's blood pressure waveform that includes these measurements. Therefore, an oximeter provides measurement values that establish an operational correlation between diastolic/systolic blood pressures, and blood volume flow values. Importantly, this correlation is applicable throughout the patient's vasculature.

Specifically, the present invention recognizes that raw data from the red and infrared diodes of a pulse oximeter can be displayed as a waveform, which is comparable to the intra-arterial blood flow, measured simultaneously in any large arterial blood vessel. This flow signal can be used to accurately estimate the blood pressure measured with a sphygmomanometer or with a direct intra-arterial pressure monitor. Therefore, oximeter flow data in its raw form, can be used to derive the blood pressure in a continuous manner, and other related parameters relating to the heart's function and tissue perfusion.

Based on the operational correlation with blood pressure measurements, waveforms provided by an oximeter can be considered indicative of blood volume flow. This operational correlation thus forms the basis for a new measurement, i.e. a tissue blood flow measurement which can be evaluated throughout the vasculature. In addition to providing a basis for new measurements, there is also a practical advantage. Specifically, unlike blood pressure measurements taken with a sphygmomanometer, which are periodic in nature, blood flow measurements taken from an oximeter are continuous. In every case, tissue blood flow measurements taken by an oximeter provide many additional ancillary measures of heart and vascular function.

Structurally, a system for measuring the blood flow of a patient in accordance with the present invention preferably includes a pulse oximeter of any type well known in the pertinent art. The importance here is that it is well known a pulse oximeter will trace changes in blood pressure during a patient's heartbeat. From such a trace the following parametric measurements can be obtained which are of specific importance. These include: 1) changes in diastolic pressure ±Δpd; 2) changes in systolic pressure, ±Δps; and 3) changes in pulse time duration, ±Δtr. Not only are these parametric measurements individually important, the comparisons of these parametric measurements relative to each other, statically and dynamically, are also important. For instance, the ratios of Δpd/Δps, Δps/Δtr, and Δpd/Δtr, as well as the cumulative values ΣΔpd, ΣΔps, and/or ΣΔtr, may be informative insofar as pressure trends are concerned. Further, the time rate of rise from pd to ps during tr is considered relative to the vigor of the heart's contractions, and the slope of the pressure runoff from ps to pd during tr is considered indicative of the peripheral vascular resistance to blood flow. In each case, regardless of whether measurements are considered in a single pulse or in a consecutive pulse-to-pulse context, comparisons of parametric measurements clearly have diagnostic value.

As part of the system for monitoring blood flow, the present invention includes a computer system that receives audiometric signals from the pulse oximeter. Importantly, these signals essentially define the blood flow waveform in the vasculature of the patient. Also included within the computer system is a calculator which uses these parametric measurements from the blood flow waveform to calculate a value for the blood flow volume in the patient's vasculature. Specifically, calculations are made for each consecutive pulse of the patient's heart muscle function. From these calculations, a blood flow volume can be considered comparable to the value of an area bounded by the blood flow waveform and a timeline underneath the blood flow waveform. In this context, for the present invention the timeline is equal in value to the time pulse rate tr of the patient's heart muscle function, e.g. the time between consecutive measurements of diastolic pressures, pd.

Included in the computer system is a monitor that receives information from the calculator to evaluate changes in the parametric measurements of a blood flow wave form. Specifically, by comparing consecutive waveforms, the changes of ±Δpd, ±Δps, and ±Δtr can be determined. Additionally, a video display is provided to present sequential values of the parametric measurements for use in evaluating the patient's health condition.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a perspective view of a system for monitoring blood flow in accordance with the present invention, with the system shown operationally connected to a patient;

FIG. 2 is a block diagram of the operative components of the system showing operational interconnections for components of the present invention;

FIG. 3 is a graph showing the pressure variations of an aortic pulse during a heartbeat of the heart muscle function;

FIG. 4 is a depiction of the essential parametric measurements used for describing a pulsed blood flow volume; and

FIG. 5 is a line graph showing variations of parametric measurements in a consecutive sequence of pulsed blood flow volumes in the context of a dynamic perspective of blood flow waveforms.

### DESCRIPTION OF THE PREFERRED EMBODIMENTS

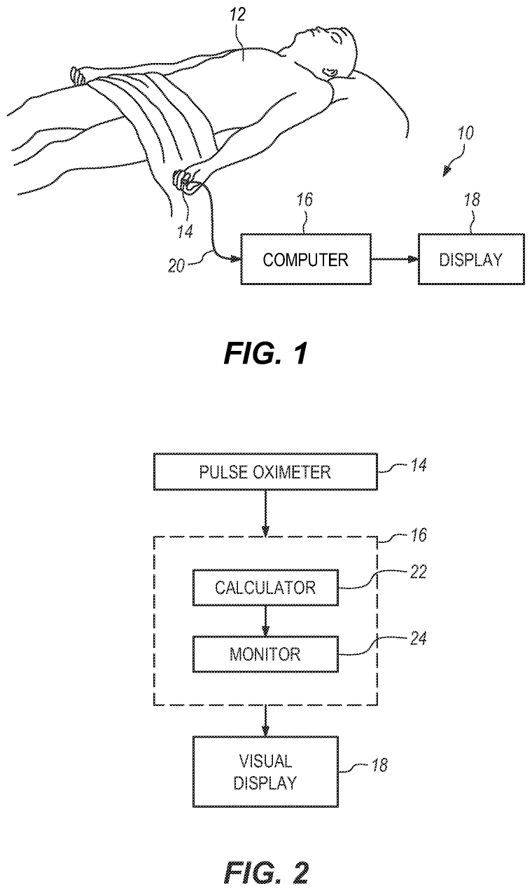

Referring initially to FIG. 1, a system for monitoring blood flow is generally designated 10. In FIG. 1 the system 10 is shown connected to a patient 12 for the purpose of measuring blood flow characteristics of the patient 12. Shown included in the system 10 is a pulse oximeter 14, a computer 16 and a visual display 18. In combination with other components of system 10, the pulse oximeter 14 is shown non-invasively positioned against the patient 12 to receive audiometric signals from the vasculature of the patient 12. Although the pulse oximeter 14 is shown positioned on a finger of the patient 12 in FIG. 1, the present invention envisions that the pulse oximeter 14 may be positioned on the patient 12 wherever positioning is clinically convenient. In any case, system 10 is intended to be electronically engaged with patient 12 via a connector 20.

In FIG. 2, the computer 16 is shown to include a calculator 22 and a monitor 24. Specifically, calculator 22 is used to receive audiometric signals from the vasculature of patient 12. With these signals, values are calculated which are based blood flow volume characteristics in the vasculature of patient 12, Specifically, the calculated values of the blood flow volume have an operational correlation with blood flow values in tissue throughout the vasculature. This is done consecutively for each pulse of the patient's heart muscle function. The monitor 24 then evaluates changes in parametric measurements of the blood flow volume as an indicator of the health condition of the patient 12. Results from this evaluation are subsequently transferred to visual display 18 for a presentation of values from the parametric measurements of the blood volume flow is provided. Clinical personnel are thereby provided with the necessary information required to accurately assess a patient's health condition.

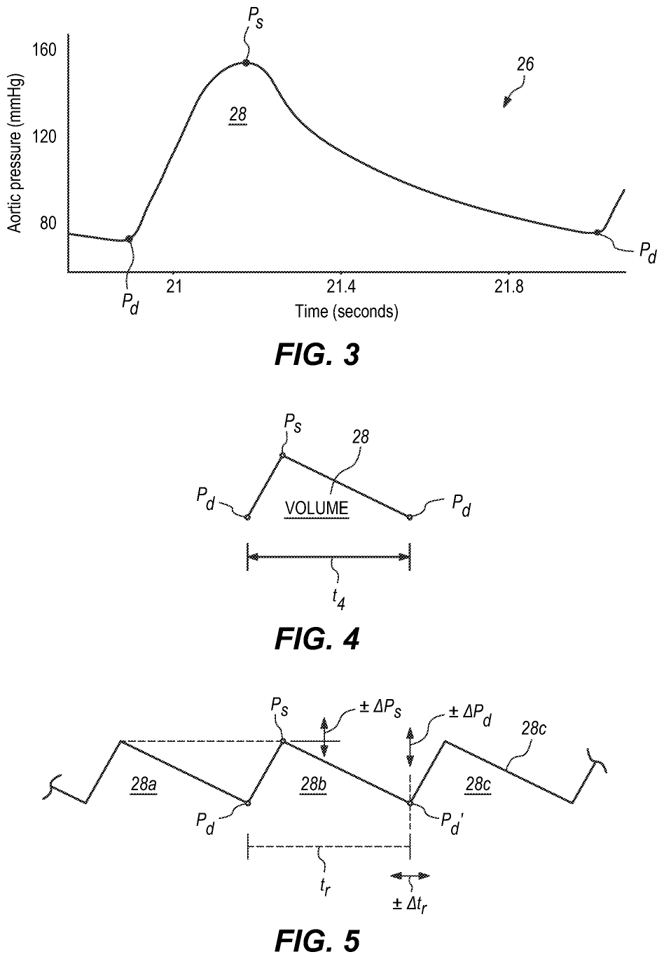

A graph 26 for a generic aortic pulse 28 is shown in FIG. 3. with annotations which illustrate and describe the time variations of aortic activities during the pulse 28. Notably, in FIG. 3 graph 26 indicates that an aortic pulse 28 can be evaluated as pressure changes in in a series of connected time segments. More specifically, as shown in FIG. 4, there is a first segment in an aortic pulse 28 that occurs during a pressure rise from a diastolic pressure, pd, to a systolic pressure ps. This first segment is then immediately followed by a second segment that occurs as the pressure falls from the systolic pressure ps to a diastolic pressure pd. At that point, another pulse 28 begins. As shown in FIG. 4, both the first and second segments of an aortic pulse 28 will occur within the pulse duration time of td.

Further, in FIG. 4 it is to be appreciated that parametric measurements from the pulse oximeter 14 can be taken to define the boundary for a blood flow volume in the vasculature of a patient 12. Specifically, for each pulse 28, the parametric measurements of diastolic pressure, pd, systolic pressure ps, and pulse time duration tr together provide reasonable values for approximating blood flow volume in the heart muscle function of patient 12 (compare FIGS. 3 and 4). It is important here to recognize that for diagnostic purposes, the dynamic values of individual parametric measurements and their variations over time alone provide valuable health information, aside from the actual blood flow volume per se. Note here also that values for the variables pd, ps and tr may vary individually or collectively from pulse to pulse.

As a technical summary for an operation of system 10 of the present invention, FIG. 5 shows a continuous sequence of pulses 28a-c which are provided for the purpose of illustrating variations in the parametric measurements being monitored. As noted above, the individual variables of diastolic pressure, pd, systolic pressure, ps, and pulse duration, tr, can be determined separately for each pulse 28 in the heart function of patient 12. It has also been noted above that each of these parametric measurements can change individually, e.g. from pulse 28a to pulse 28b, et seq. With specific reference to the pulse 28b, note that in comparison with the previous pulse 28a, it is possible that a change of systolic pressure equal to ±Δps may have occurred. Further, it is also noted that during the pulse 28b, the diastolic pressure pd at the beginning of the pulse 28b may change to pd' at the end of the pulse 28b. Thus, there is a change in pressure equal to ±Δpd during the pulse 28b. It can also happen that the time duration tr will change during between consecutive pulses 28, with an increase or decrease equal to ±Δtr. Accordingly, there are many variations in parametric measurements that may have pertinent information for a further analysis of a health condition.

While the system and method for measuring blood flow in a patient as herein shown and disclosed in detail are fully capable of obtaining the objects and providing the advantages herein before stated, it is to be understood that they are merely illustrative of the presently preferred embodiments of the invention and that no limitations are intended in the details of construction or design herein shown other than as described in the appended claims.

What is claimed is:

1. A system for monitoring blood flow in a patient which comprises:
a pulse oximeter adapted to be non-invasively positioned against the body of a patient to receive audiometric signals from the vasculature of the patient, wherein the received signals are descriptive of a blood flow waveform;
a computer system having a readable medium with executable instructions stored thereon that direct the computer system to collect parametric measurements of the blood flow waveform;
a calculator included in the computer system for using the parametric measurements of the waveform to calculate a value for a blood flow volume in the patient's vasculature during each pulse of the patient's heart muscle function; and
a monitor connected to the computer system to evaluate changes in the parametric measurements of blood flow volume as an indicator of the patient's health condition.

2. The system of claim 1 wherein the calculated values of the blood flow volume have an operational correlation with blood flow values in tissue throughout the vasculature which can be used for diagnostic purposes.

3. The system of claim 2 wherein the parametric measurements of the waveform include, in sequence:
a diastolic blood pressure measurement, p_{d}:
a systolic blood pressure measurement, pₛ: and a time pulse rate (seconds/pulse), tᵣ, for each pulse of the patient's heart muscle function.

4. The system of claim 3 wherein each blood flow volume is comparable to the value of an area bounded by the blood flow waveform and a timeline underneath the blood flow waveform equal in value to the time pulse rate tᵣ of the patient's heart muscle function.

5. The system of claim 4 wherein the blood flow waveform is a trace of changes in blood pressure measurements in a sequence of diastolic-systolic-diastolic measurements during each pulse of the patient's heart muscle function.

6. The system of claim 5 wherein the timeline for each pulse of the patient's heart muscle function includes when the patient's aortic valve opens and when it closes.

7. The system of claim 6 wherein the monitor evaluates respective changes in a diastolic pressure, ±Δp_{d}, a systolic pressure ±Δpₛ, and ±Δtᵣ.

8. The system of claim 7 wherein the change in each parametric measurement is evaluated in comparison with the change in every other parametric measurement during tᵣ.

9. The system of claim 8 wherein indicators of a patient's health condition include changes in the blood flow volume, based on changes in blood pressure measurements ±Δp_{d}, and ±Δpₛ, and changes in the time pulse rate ±Δtᵣ.

10. A method for monitoring blood flow in a patient which comprises the steps of:
receiving audiometric signals from the vasculature of the patient, wherein the received signals are descriptive of a blood flow waveform;
collecting parametric measurements of the blood flow waveform;
identifying values for the parametric measurements from the blood flow volume waveform in the patient's vasculature during each pulse of the patient's heart muscle function; and
evaluating changes in the calculated parametric measurements as an indicator of the condition of the patient's cardio vascular system.

11. The method of claim 10 wherein values of the blood flow volume have an operational correlation with blood flow values in tissue throughout the vasculature which can be used for diagnostic purposes.

12. The method of claim 11 wherein the parametric measurements of the waveform sequentially include:
a diastolic blood pressure measurement, p_{d}:
a systolic blood pressure measurement, pₛ: and
a time pulse rate (seconds/pulse), tᵣ, for each pulse of the patient's heart muscle function.

13. The method of claim 12 wherein the blood flow waveform is bounded by a timeline underneath the blood flow waveform equal in value to the time pulse rate tᵣ of the patient's heart muscle function.

14. The method of claim 13 wherein the blood flow waveform is a trace of changes in blood pressure measurements in a sequence of diastolic-systolic—diastolic measurements during each pulse of the patient's heart muscle function, and wherein the timeline for each pulse of the patient's heart muscle function extends between the diastolic pressure at the beginning of the pulse and the diastolic pressure at the end of the pulse.

15. The method of claim 14 wherein the change in each parametric measurement is evaluated in comparison with the change in every other parametric measurement during tᵣ, and wherein indicators of a patient's health condition are based on changes in blood pressure measurements ±Δp_{d}, and ±Δpₛ, and changes in the time pulse rate ±Δtᵣ.

16. A system for monitoring blood flow in a patient which comprises:
a means adapted to be non-invasively positioned against the body of a patient for receiving audiometric signals from the vasculature of the patient, wherein the received signals are descriptive of a blood flow waveform;
a means having a readable medium with executable instructions stored thereon for directing the receiving means to collect parametric measurements of the blood flow waveform;
a means for using the parametric measurements of the waveform to calculate values for changes in the blood flow waveform in the patient's vasculature during each pulse of the patient's heart muscle function wherein the calculated values of the blood flow volume have an operational correlation with blood flow values in tissue throughout the vasculature which can be used for diagnostic purposes;
a means for evaluating changes in the blood flow waveform as indicators of the patient's health condition; and
a means for presenting sequential values of parametric values on a visual display for use in evaluating the patient's health condition.

17. The system of claim 16 wherein the parametric measurements of the waveform sequentially include:
a diastolic blood pressure measurement, p_{d};
a systolic blood pressure measurement, pₛ; and
a time pulse rate (seconds/pulse), tᵣ, for each pulse of the patient's heart muscle function.

18. The system of claim 17 wherein each blood flow waveform is bounded by a timeline underneath the blood flow waveform, wherein the timeline is equal in value to the time pulse rate tᵣ of the patient's heart muscle function.

19. The system of claim 18 wherein the blood flow waveform is a trace of changes in blood pressure measurements in a sequence of diastolic-systolic—diastolic measurements during each pulse of the patient's heart muscle function, and wherein the timeline for each pulse of the patient's heart muscle function extends between the diastolic pressure at the beginning of the pulse and the diastolic pressure at the end of the pulse.

20. The system of claim 19 wherein the evaluating means compares respective changes in a diastolic pressure, ±Δp_{d}, a systolic pressure ±Δpₛ, and ±Δtᵣ, which are each evaluated in comparison with one another during tᵣ as indicators of a patient's health condition.
